(19) United States
(12) Patent Application Publication
(10) Pub. No.: US 2002/0177544 A1
(43) Pub. Date: Nov. 28, 2002

(54) ADENOVIRAL TRANSFER VECTOR FOR THE GENE TRANSPORT OF A DNA SEQUENCE

(75) Inventors: ANJA HAACK, GRAFSCHAFT (DE); CHRISTOPH SCHMITT, GRAFSCHAFT (DE)

Correspondence Address: FINNEGAN HENDERSON FARABOW, GARRETT AND DUNNER, FRANKLIN SQUARE BUILDING SUITE 700, 1300 I STREET NW, WASHINGTON, DC, 200053315

(73) Assignee: AVENTIS BEHRING GMBH

(21) Appl. No.: 09/252,819

(22) Filed: Feb. 19, 1999

(30) Foreign Application Priority Data
Feb. 20, 1998 (DE) 198 07 265.1

(51) Int. Cl. A61K 48/00; A61K 38/17; C12P 21/02; C12N 15/861

(52) U.S. Cl. 514/2; 514/44; 514/834; 435/69.1; 435/70.1; 435/455; 435/456; 435/457; 435/320.1; 536/23.1; 536/23.5; 536/23.2

(57) ABSTRACT

An adenoviral transfer vector for the gene transport of a DNA sequence, which is produced from an adenoviral plasmid which no longer expresses any natural adenoviral proteins and comprises

a) a first DNA sequence with the left inverted terminal repeat (ITR) sequence and a packaging signal of the wild-type adenovirus (serotype 5) and

b) a second DNA sequence with the right inverted terminal repeat (ITR) sequence of the wild-type adenovirus (serotype 5) and

c) cleavage sites for restriction endonucleases which do not occur in the therapeutic genes and/or marker genes to be incorporated between the adenoviral DNA sequences, and preferably

d) the ITRs are enclosed by cleavage sites of a restriction endonuclease which cuts but rarely (i.e. the recognition sequence in ≧8 base pairs), preferybly Fsel, which makes it possible to cut out the adenoviral portion of the transfer vectors, is described.

The invention relates to an adenoviral transfer vector suitable for gene transport, and to a combination product which comprises a transfer vector and can be employed for treating classical hemophilia A.

It is known that many disorders are caused by genetic defects. In these cases, there are prospects of a permanent cure only if it is possible to replace the defective or missing gene in the body. This is the reason for the intensive search for methods for somatic gene therapy which, through a single administration, or an administration repeated at long intervals of time, of the intact gene lead to restoration of the patient's health. One example of these efforts are the attempts to achieve a cure of hemophilia A by somatic gene therapy.

Hemophilia A is caused by a chromosomal defect which results in a deficiency of factor VIII which is required for coagulation of blood. The genetic defect is located in the X chromosome and is transmitted through the woman's genetic material, although the women carriers are not themselves hemophiliacs. Promising treatment of hemophilia A became possible only when plasma concentrates of factor VIII became available. With factor concentrates from pooled plasma from human donors there is in principle the possibility that previously unknown or not reliably identifiable infectious agents are also transmitted, although the inactivation methods which have now been introduced, and the individual purification steps themselves, inactivate or remove such agents. The use of recombinant human coagulation factors produced in eukaryotic cells very considerably reduces any risk. However, it is a disadvantage that expression of wild-type factor VIII has to date been possible only in comparatively small amounts. This is explained, on the one hand, by the fact that the factor VIII molecule is an unusually large polypeptide with 2332 amino acids, and the mRNA concentrations are relatively low in the expression systems employed to date therefor. In addition, the transport of the initial translation product from the endoplasmic reticulum to the Golgi apparatus evidently does not take place very efficiently, which is reflected, because of the low secretion rates, by low yields in the cell culture supernatant.

This is why there have already been investigations with the aim of truncating the cDNA of factor VIII. The International Patent Applications WO 86/06101, WO 87/07144 and WO 91109122 have already reported on the recombinant production of truncated factor VIII derivatives which have the same biological effect as the wild-type factor VIII protein but are expressed in considerably larger amounts in animal cell cultures. Thus, for example, J. J. Toole et al. have reported the production and expression of factor VIII derivatives lacking amino acids 982 to 1562 or amino acids 760 to 1639 (Proc. Natl. Acad. Sci. USA (1986) 83, 5939-5942). D. L. Eaton et al. reported on the production and expression of a factor VIII derivative lacking amino acids 797 to 1562 (Biochemistry (1986) 25, 8343 to 8347). R. J. Kaufman described the expression of a factor VIII derivative lacking amino acids 741 to 1646 (International Patent Application WO 87/04187). N. Sarver et al. reported on the production and expression of a factor VIII derivative lacking amino acids 747 to 1560 (DNA (1987) 6, 553 to 564). M. Pasek reported on the production and expression of factor VIII derivatives lacking amino acids 745 to 1562 or amino acids 741 to 1648 (International Patent Application WO 88/00831). K.-D. Langner reported on the production and expression of factor VIII derivatives lacking amino acids 816 to 1598 or amino acids 741 to 1689 (Behring Inst. Mitt., (1988) No. 82, 16 to 25, European Patent Application 0 295 597). P. Meulien et al. reported on the production and expression of factor VIII derivatives lacking amino acids 868 to 1562 or amino acids 771 to 1666 (Protein Engineering (1988), 2 (4), 301 to 306, European Patent Application 0 303 540). On expression of these truncated factor VIII derivatives in mammalian cell lines the expression of factor VIII was usually found to be ten times greater than production of wild-type factor VIII.

It has repeatedly been observed on expression of truncated factor VIII derivatives that the highly glycosylated B domain of the factor VIII molecule between amino acids Arg-739 and Glu-1649 is unnecessary for the procoagulant and cofactor activity. This realization has prompted the search for further truncated factor VIII derivatives which can be expressed in high yields, show the full biological effect of factor VIII and, moreover, can be incorporated into suitable vectors which make somatic gene therapy of hemophilia A possible. Somatic gene therapy currently appears to be the only method of treating hemophilia A with which it is possible to dispense with continual administration of exogenous factor VIII, which remains effective in the recipient's body only for a short half-life. By contrast, somatic gene therapy would make it possible to ensure adequate production of factor VIII permanently through a single administration or through repeated administrations at relatively long intervals of time (several weeks or years).

Various gene transfer systems have been developed in recent years and are associated with many hopes that they can be used for somatic gene therapy.

One of the most promising systems comprises the so-called replication-defective adenoviral vectors which ensure a very high gene transfer efficiency, not only ex vivo but also in vivo in the intact whole organism, which cannot be achieved with any other currently available system. A vector system of this type has already been described in European Patent Application 0 808 905.

However, it is not possible to incorporate either the wild-type factor VIII cDNA or a truncated factor VIII cDNA coding for a biologically active factor VIII derivative into this specific vector system because they are too large.

However, the International Patent Application WO 96133280 has already disclosed an adenoviral helper virus system able to take up as much as 36 kB of a heterologous DNA in an adenoviral vector no longer capable of replication. This system consists of an adenoviral vector construct, one or more helper viruses and a helper cell line. The vector construct can be replicated and a virion particle can be packaged in the helper cell line if it is administered together with a helper virus which contains a defective packaging signal. The object therefore was to modify and improve an adenoviral transfer system of this type in such a way that it can be used in somatic gene therapy for transferring genes which can be employed in therapy.

The invention thus relates to an adenoviral transfer vector for the gene transport of a DNA sequence, which vector is produced from an adenoviral plasmid which no longer expresses natural adenoviral proteins and comprises

a) a first DNA sequence with the left inverted terminal repeat (ITR) sequence and a packaging signal of the wild-type adenovirus (serotype 5) and

b) a second DNA sequence with the right inverted terminal repeat (ITR) sequence of the wild-type adenovirus (serotype 5) and

c) cleavage sites for restriction endonucleases which do not occur in the therapeutic genes and/or marker genes to be incorporated between the adenoviral DNA sequences, where preferably

d) the ITRs are enclosed by cleavage sites of a restriction endonuclease which cuts but rarely (i.e. the recognition sequence in ≧8 base pairs), preferybly Fsel, which makes it possible to cut out the adenoviral portion of the transfer vector.

Deletion of all the adenoviral genes results in a packaging capacity of up to 36 kb. If it is wished to cut such a DNA fragment integrated into a plasmid out of the latter, there is a need for flanking cleavage sites for restriction endonucleases which cut very rarely. The recognition sequences of these endonucleases are usually 4, 6 or 8 base pairs long. Viewed statistically, an endonuclease which recognises a 6 bp-long recognition sequence cuts once every 4096 base pairs, but at 8 bp cuts only once every 65536 base pairs. In order to have the maximum number of different possibilities for cloning different DNA fragments into such a minivector construct, it is sensible to use an enzyme which cuts very rarely so that inserted DNA fragments are not cut up when the minivector DNA is cut out.

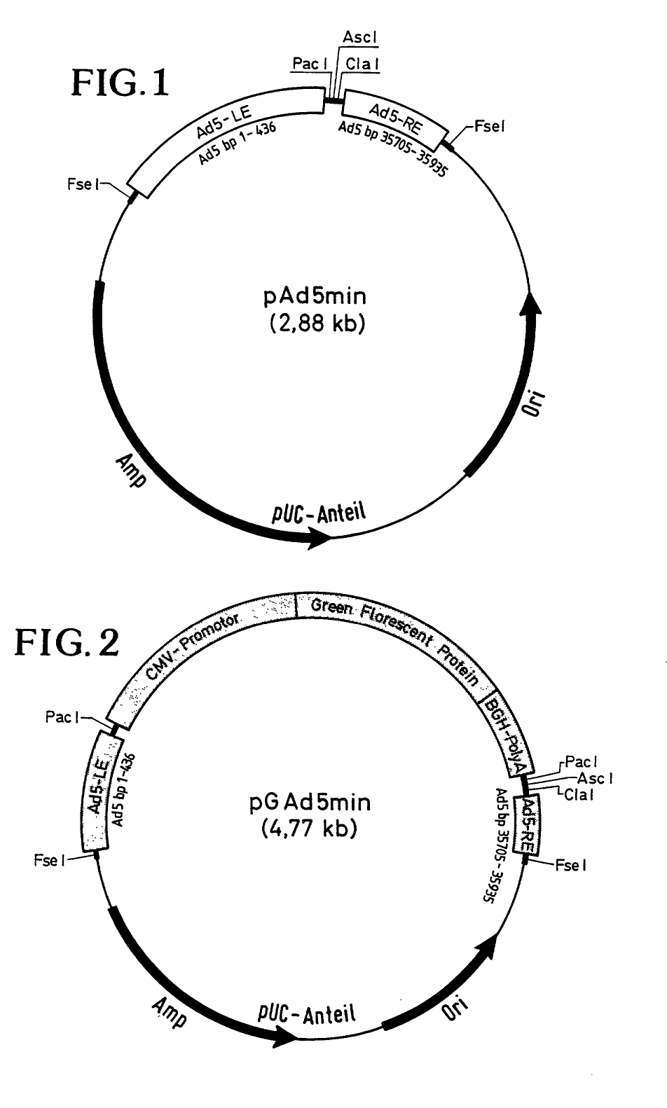

The plasmid pAd5min depicted in FIG. 1 is an example of an adenoviral transfer vector of this type. The sequences of the plasmid pUC19 to which is attached, via the Fsel cleavage site, initially the left inverted terminal repeat sequence and a packaging signal (ITR and packaging signal=Ad5-LE) with base pairs 1 to at least 358 of the wild-type adenovirus (serotype 5). Pacl, Ascl and Clal cleavage sites then follow and are connected to the DNA sequence of the right inverted terminal repeat sequence (ITR=Ad5-RE) consisting of base pairs 35,705 to 35,935, but at least of base pairs 35833 to 35935, are depicted. Another Fsel cleavage site is then inserted. This plasmid construct allows the Ad5 portion to be cut out by means of the enzyme Fsel. The resulting linear DNA contains the same ends as the linear adenovirus wild-type, extended by one cytosine base on each side.

Fse I has an 8 bp-long recognition sequence and, for example, does not cut factor VIII cDNA. This means that it is possible to cut out the minivector DNA including factor VIII cDNA and any further gene/DNA sequences without cutting up the sequences located between the two limiting cleavage sites.

For the same reason, a usual multiple cloning site (MCS) between the adenoviral ends was also dispensed with.

The problem with a usual MCS is that many cleavage sites of the MCS also occur at least once in factor VIII. This means that it is no longer possible, after inserting the factor VIII cDNA into the MCS, to insert a second sequence into the MCS without at the same time cutting the factor VIII. Insertion of a second sequence between the adenoviral ends would thus be impossible. The Asc I cleavage site (8 bp recognition sequence) present in the plasmid construct according to the invention does not cut the factor VIII sequence and, moreover, cuts very rarely in all other sequences. Attachment of DNA fragments which have been cut on one side with Asc I and on the other with Mlu I (Mlu I generates the same protruding ends as Asc I) to this Asc I cleavage site again results, after ligation, in an Asc I cleavage site in the minivector construct. This site can be used in this way several times in succession without cutting up the factor VIII cDNA or other inserted sequences.

A specific plasmid for synthesizing the Mlu I/Asc I fragments has therefore been constructed according to the invention. This has an expression cassette into which genes or cDNAs can be inserted via an MCS. The complete expression cassette can be cut out with Mlu I and Asc I and inserted at the Asc I site in the minivector construct.

Pac I is also an enzyme which cuts rarely (8 bp recognition sequence), which makes it possible to cut out the GFP sequence, which is not required for in vivo tests, without cutting up the other DNA sequences.

Thus, in summary, it can be stated that the vector according to the invention has been constructed so that incorporation of marker genes, for example for the green fluorescent protein (GFP), or of therapeutic genes, such as those for factor VIII and factor IX or, for example, immunomodulating adenovirus genes of the E3 region, is easily possible. The restriction endonucleases Pacl, Ascl and Fsel have individual cleavage sites which are each 8 bp long and therefore cut only very rarely. This means that the Pacl and Ascl cleavage sites permit a wide variety of genes to be inserted, because they are not cut by the restriction endonucleases. Although Clal cuts more frequently, it is distinguished by cutting only once in the adenovirus. Pacl has the advantage that it is very suitable for cloning the GFP marker gene, whereas Ascl and Clal make it possible repeatedly to connect various genes in series via the same cleavage sites. The Ascl cleavage site allows a DNA fragment to be cloned at this cleavage site if the DNA fragment has an Mlul cleavage site in the 5′ position and an AscI cleavage site in the 3′ position, with an AscI cleavage site being produced once again. Cloning at the Clal cleavage site is possible similarly. The rare occurrence of Fsel cleavage sites in the DNA makes it possible to cut the adenovirus portion together with the inserted genes out of the complete plasmid.

It is possible to insert expression cassettes with up to about 35 kb DNA into an adenoviral transfer vector of this type between the two adenoviral ends. This makes it possible, for example, to incorporate the complete or a truncated cDNA sequence of factor VIII and, where appropriate, further expression cassettes with immunomodulating and/or DNA stabilizing genes into the virus construct.

FIG. 2 shows an example of a transfer vector according to the invention, in which an expression cassette consisting of the cytomegalovirus (CMV) promoter, the GFP and the bovine growth hormone (BGH) polyA-DNA.

This plasmid construct is called pGAd5min and, owing to the rare occurrence of the Pacl cleavage site, the FGP gene can be cut out of it at later times, even after cloning of other genes.

The production of recombinant viruses necessitates simultaneous transfection with a (HEK-293) cell line and with an adenoviral transfer vector of this type (for example pGAd5min) and with a helper virus or a helper plasmid. The transfer vector pAd5min and its derivatives do not express any adenoviral proteins unless these have been subsequently inserted. The proteins necessary for the synthesis of adenoviruses must therefore be provided in trans. The wild-type virus is preferably used for this. After production of the viruses in cell culture, they must be purified and separated by a cesium chloride gradient.

The abovementioned empty transfer vector is particularly suitable for incorporating a cDNA which codes for a polypeptide with amino acids 1 to 852 and 1524 to 2332 of human factor VIII.

The cDNA sequence necessary for producing this factor VIII peptide was obtained from the cDNA of the complete factor VIII (ATCC 39812-pSP64-VIII) by cloning in pBluescript II KS(-) phagemid (Stratagene) using the restriction enzyme Sal I. The pKS-FVIII resulting thereby was cleaved with the restriction enzyme EcoNI (New England Biolabs) and, in this way, the DNA section coding for amino acids 853 to 1523 was deleted. Subsequently, the non-complementary DNA ends were linked to the oligonucleotide linker sequence described in Example 1. Correct incorporation of the linker fragment was verified by sequencing. The cDNA produced in this way for truncated factor VIII was subsequently inserted into the expression plasmid pCI-neo (Promega) using Sall restriction sites. Expression took place in Chinese hamster ovary (CHO), monkey kidney (COS) and human embryonic kidney (HEK-293) cell lines.

For comparison, the cDNA of the wild-type factor VIII gene was also inserted into the same expression systems.

It was possible to demonstrate that the truncated factor VIII polypeptide has the same biological activity as wild-type factor VIII. At the same time, expression rates were observed to differ in the different expression media but were in most cases considerably higher than the expression rate of wild-type factor VIII. It was additionally possible to show that the truncated factor VIII polypeptide has increased stability and can be concentrated in the expression medium, which indicates that the truncated factor VIII has considerably less sensitivity to proteolytic degradation. This is a marked difference from wild-type factor VIII, with which considerable losses are observed due to proteolytic degradation in the expression medium. Deletion of the B domain of the factor VIII molecule, the biological function of which it has not to date been possible to elucidate, thus not only increases the yield but appears also to improve the stability of the truncated factor VIII derivative toward proteases.

The abovementioned properties of the truncated factor VIII polypeptide are advisable prerequisites for incorporation of the coding cDNA into a transfer vector according to the invention which can be employed for somatic gene therapy. It is moreover desirable to introduce the cDNA for the modified factor VIII into the liver, the physiological site of synthesis of factor VIII. The publication in J. Biol. Chem. 265, page 7318 et seq. (1990) has already disclosed the expression of a factor VIII capable of functioning in fibroblasts from human skin, the gene transfer having been carried out with a retroviral vector system. However, this vector system is unsuitable for introducing the factor VIII gene into the liver. By contrast, the abovementioned adenoviral vector is ideal for gene transfer into the liver. By using an adenovirus modulated in this way and using this vector for gene transfer with simultaneous transient anti-CD4 treatment of the recipient organism might stabilize the expression of the factor VIII cDNA at a high level over a long period. The anti-CD4 treatment is preferably carried out with suitable monoclonal antibodies against CD4 antigens overlapping in time with the administration of the adenoviral vector for the gene transfer. The essential element of this transfer system is the combination of the E3-positive vector with the anti-CD4 strategy for improving hepatic gene transfer.

Suitable monoclonal anti-CD4 antibodies are those which block signal transduction from the CD4 receptor or deplete CD4-positive lymphocytes from the target organism. Particularly preferred in each case are corresponding humanized monoclonal antibodies. This adenoviral vector system makes it possible to transfer the cDNA according to the invention into the liver as target organ and, in combination with transient anti-CD4 treatment, to ensure considerably improved tolerance.

The invention is explained in detail by the following examples:

### EXAMPLE 1

Production of a Truncated cDNA for Factor VIII

The cDNA for factor VIII obtained from the strain ATCC 39812-pSP64-VIII was cloned in pBluescript II KS(-) phagemid (Stratagene) using the Sall restriction sites. The plasmid pKS-FVIII resulting from this was cut with Eco NI (New England Biolabs), and the non-complementary ends were connected together by inserting oligonucleotide linkers comprising eleven base pairs. The following oligonucleotides were used for this

| | |
|---|---|
| SEQ ID No. 1: | ^{5′} G TCA GGC CTC C ^{3′} |
| SEQ ID No. 2: | ^{5′} AG GAG GCC TGA ^{3′} |

Correct insertion of the linkers was demonstrated by automatic fluorescence sequence analysis (Applied Biosystems 373 A). The resulting truncated cDNA (5.2 kb) codes for a truncated factor VIII protein in which amino acid 852 is connected to amino acid 1524.

### EXAMPLE 2

Expression Systems

The wild-type and the truncated factor VIII cDNA were cloned into the expression plasmid pCI-neo (Promega) using the Sall restriction sites. CHO, COS and HEK-293 cell lines were used for the expression. The cell medium consisted of 10% fetal calf serum (BioWhitaker, 1% penicillin/streptomycin solution (BioWhitaker) and Hams F12 (Gibco) for CHO cells or Dulbecco's modified Eagle medium (Gibco) for HEK-293 and COS cell lines.

The transfection was carried out on six microtiter plates (Nunc) at a cell density of about 70%. 15 μl of lipofectamine reagent (Gibco), 1.5 μg of endotoxin-free plasmid DNA (purified with the Qiagen plasmid kit, Endo free maxi) and 1 ml of serum-reduced medium (OptiMEM, Gibco) were mixed in each well of the microtiter plate. After incubation for 30 minutes, the transfection mixture was added to the cells and then incubated for up to 24 hours. The transfection was then stopped by collecting the mixture and immediately incubating the cells with normal medium.

### EXAMPLE 3

Factor VIII Antigen Determination

The antigen determination was carried out with a high-sensitivity factor VIII ELISA. In this sandwich ELISA, the plates were coated with a monoclonal anti-human factor VIII antibody (ESH5, American Diagnostica). The bound factor VIII was detected with a polyclonal sheep anti-human factor VIII antibody (Cedarlane) and a polyclonal, peroxidase-coupled, donkey anti-sheep IgG antibody (Jackson ImmunoResearch Laboratories). Pooled plasma from normal individuals was used as standard.

The factor VIII activity was measured by a chromogenic assay (DADE® factor VIII chromogen, Baxter), using both a one-stage assay based on natural, factor VIII-free plasma and a one-stage assay based on plasma free of factor VIII due to immunoadsorption (Immuno).

### EXAMPLE 4

Construction of a Completely Deleted Adenoviral Transfer Vector

231 bp of the 3′ end, based on the I strand of the wild-type adenovirus (serotype 5), were multiplied by PCR, employing the primers Ad5-RE-A and Ad5-RE-B with the following base sequences:

| | |
|---|---|
| Base sequence of the primer Ad5-RE-A | (= SEQ ID No. 3): |
| ^{5′} TTG GCG CGC CAT CGA TGC CCA GAA ACG AAA GCC AAA AAA | |
| CCC ^{3′} | |
| Base sequence of the primer Ad5-RE-B | (=SEQ ID No. 4): |
| ^{5′} CCC AAG CTT GGC CGG CCA TCA TCA ATA ATA TAC CTT ATT TTG | |
| G ^{3′} | |

This resulted in attachment to the PCR product of an Ascl and a Clal restriction enconuclease cleavage site in the 5′ position and an Fsel and a Hind III cleavage site in the 3′ position. It was possible to clone the PCR product into the plasmid pNEB193 (New England Biolabs) via the Ascl and the Hind III cleavage sites. The resulting plasmid was called pUCRE.

436 bp of the adenovirus 5′ end was amplified analogously with the primers Ad5-LE-A2 and Ad5-LE-B2.

| | |
|---|---|
| Base sequence of the primer Ad5-LE-A2 | (= SEQ ID No. 5): |
| ^{5′} GGG ACG TCG GCC GGC CAT CAT CAA TAA TAT ACC TTA TTT | |
| TGG ^{3′} | |
| Base sequence of the primer Ad5-LE-B2 | (= SEQ ID No. 6): |
| ^{5′} TTG ACG TCG GCG CGC CTT AAT TAA CGC CAA CTT TGA CCC | |
| GGA AGG C ^{3′} | |

It was possible to clone this PCR product into pUCRE via AatII and AscI by means of the attached cleavage sites for the enzyme AatII and Fsel at the 5′ end, and Pacl and AscI at the 3′ end.

This resulted in the plasmid depicted in FIG. 1. This plasmid construct allows the Ad5 portion to be cut out with the enzyme Fsel. The resulting linear DNA contains the same ends as the linear adenovirus wild-type extended by one cytosine base on each side.

### EXAMPLE 5

Incorporation of an Expression Cassette in the Plasmid pAd5min

The DNA for the green fluorescent protein (GFP) was clonsed from pEGFP-N1 (Clontech) via BamHi and Xbal cleavage sites into pABS.4 (Microbix). The promoter and the polyA signal were cloned from pRc/CMV (Invitrogen) via the Apol/EcoRI and Kpal or Xbal and Xhol/Sall cleavage sites into the above construct. The plasmid was called pAGFP/CB. It was possible for GFP together with the expression cassette and kanamycin resistance to be isolated from this plasmid by means of the Pacl cleavage sites and cloned in pAd5min. It was then possible to delete the resistance from the plasmid via Sawl cleavage sites. This plasmid construct was called pGAd5min. Because of the rare occurrence of the Pacl cleavage site it is always possible for the GFP gene to be cut out of the plasmid at later times and after cloning of other genes.

Patent claims for USA:

1. An adenoviral transfer vector for the gene transport of a DNA sequence, which is produced from an adenoviral plasmid which no longer expresses any natural adenoviral proteins and comprises
a) a first DNA sequence with the left inverted terminal repeat (ITR) sequence and a packaging signal of the wild-type adenovirus (serotype 5) and
b) a second DNA sequence with the right inverted terminal repeat (ITR) sequence of the wild-type adenovirus (serotype 5) and
c) cleavage sites for restriction endonucleases which do not occur in the therapeutic genes and/or marker genes to be incorporated between the adenoviral DNA sequences, and preferably
d) the ITRs are enclosed by cleavage sites of a restriction endonuclease which cuts but rarely (i.e. the recognition sequence in >8 base pairs), preferably Fsel, which makes it possible to cut out the adenoviral portion of the transfer vector.

2. A transfer vector as claimed in claim 1 wherein the first DNA sequence comprises base pairs 1 to at least 358 and the second DNA comprises base pairs 35705-35935, but at least base pairs 35833 to 35935, of the wild-type adenovirus (serotype 5).

3. A transfer vector as claimed in claim 1, which comprises in each case a cleavage site for the restriction endonucleases Clal and/or Ascl, which makes possible repeated attachment of identical or different cDNA sequences in series at the same cleavage site.

4. A transfer vector as claimed in claim 1, which comprises an expression cassette with the complete cDNA sequence, or a truncated cDNA sequence coding for amino acids 1-852 and 1524-2332, of human factor VIII and, where appropriate, other expression cassettes with immunomodulating and/or DNA-stabilizing genes.

5. A transfer vector as claimed in claim 1, which additionally comprises one or more marker genes enclosed by cleavage sites suitable for deletion from the vector.

6. A transfer vector as claimed in claim 1, which has been constructed with the aid of an adenoviral helper virus or helper plasmid and a helper cell line.

7. The use of a transfer vector as claimed in claim 1 for producing a pharmaceutical which can be employed for somatic gene therapy.


SEQUENCE LISTING (6 sequences)

SEQ ID NO: 1 - DNA, 11 bp, Artificial Sequence
  Description of Artificial Sequencelinker
gtcaggcctcc

SEQ ID NO: 2 - DNA, 11 bp, Artificial Sequence
  Description of Artificial Sequencelinker
aggaggcctga

SEQ ID NO: 3 - DNA, 42 bp, Artificial Sequence
  Description of Artificial Sequenceprimer
ttggcgcgccatcgatgcccagaaacgaaagccaaaaaaccc

SEQ ID NO: 4 - DNA, 43 bp, Artificial Sequence
  Description of Artificial Sequenceprimer
cccaagcttggccggccatcatcaataatataccttattttgg

SEQ ID NO: 5 - DNA, 42 bp, Artificial Sequence
  Description of Artificial Sequenceprimer
gggacgtcggccggccatcatcaataatataccttattttgg

SEQ ID NO: 6 - DNA, 46 bp, Artificial Sequence
  Description of Artificial Sequenceprimer
ttgacgtcggcgcgccttaattaacgccaactttgacccggaacgc